(19) United States
(12) Patent Application Publication
(10) Pub. No.: US 2025/0009964 A1
(43) Pub. Date: Jan. 9, 2025

(54) SYSTEMS AND METHODS FOR OPERATION DELEGATION FOR INSULIN PUMP SYSTEMS

(71) Applicant: Tandem Diabetes Care, Inc., San Diego, CA (US)

(72) Inventors: Geoff KRUSE, San Diego, CA (US); Josh JUSTER, San Diego, CA (US)

(21) Appl. No.: 18/749,369

(22) Filed: Jun. 20, 2024

Related U.S. Application Data
(60) Provisional application No. 63/511,831, filed on Jul. 3, 2023.

(51) Int. Cl. A61M 5/142; G16H 40/67

(52) U.S. Cl. CPC A61M 5/14248; G16H 40/67; A61M 2005/14208

(57) ABSTRACT

Systems and methods are provided including insulin pump systems having a wearable insulin pump that may delegate one or more operations to a periphery device and/or remote device for remote execution. For example, the insulin pump may be in wireless communication with a mobile device (e.g., mobile phone) and/or a smart device (e.g., smart watch), which may be in wireless communication with a remote device (e.g., remote server). Insulin pump may determine to send instructions to a periphery device or remote device to perform an operation on such device and to generate an outcome or output based on the operation, which will be sent back to the insulin pump. The insulin pump may then adjust pump operation based on the outcome or output generated by the operation.

### CROSS-REFERENCE TO RELATED APPLICATION

This application claims priority to U.S. Patent Application Ser. No. 63/511,831, filed on Jul. 3, 2023, the entire contents of which is incorporated herein by reference.

### TECHNICAL FIELD

This technology generally relates to the field of medication delivery pumps including pumps for delivering insulin to a user. For example, systems and methods are provided herein for insulin pumps worn by a user in wireless communication with a periphery device.

### BACKGROUND

Nearly 1 in 10 individuals in the United States are affected by diabetes. As technology advances, techniques for glucose monitoring and even insulin delivery so too develop and evolve. To manage the condition, historically many of these individuals were required to administer a blood draw, for example a need prick in the fingertip to analyze the blood to determine blood glucose levels. If the blood glucose level did not satisfy a threshold, the individual may have to administer an insulin shot, meaning an injection of insulin into the subcutaneous tissue using a needle and syringe.

With advances in monitoring technology, continuous glucose monitoring using a wearable patch including a small needle provides on demand glucose monitoring without the need for multiple needle pricks throughout the day. Additionally, technological advances in pump technology and insulin administration have resulted in wearable pumps and even patch pumps adhered to a user. With the wearable pump, the individual is no longer required to inject insulin using a need and a syringe. Instead the pump will deliver insulin at the direction of the user or may even automatically deliver insulin (e.g., based on a schedule or input from a glucose monitor).

While today's insulin pumps are a significant improvement over the traditional approach involving periodic insulin injections using a needle and a syringe, such user worn pumps introduce new complexities and challenges such as managing battery power. Additionally, it is desirable for such wearable pumps to be small in size and light in weight and thus hardware and battery size and weight are limited. For this reason, computing power, memory storage, and available power are often limited, which may impact pump operation and function. For example, the computational operations that may be performed by the pump may be limited.

Accordingly, there is a need for improved systems and methods for conserving energy and achieving complex computational operations and other demanding tasks using a wearable insulin pump.

### SUMMARY

Provided herein are systems and methods for an insulin pump system including an insulin pump and one or more periphery and/or remote devices. The insulin pump may be a wearable pump or a pump patch and may be in communication with the periphery and/or remote devices. As the insulin pump may have limited processing power and limited battery life, the insulin pump may send operations (e.g., operations, tasks, analysis, calculation, computations, etc.) to a periphery device or a remote device for such device to perform the operation and to generate outcome or output data. The outcome or output data may be sent back to the insulin pump and may include adjusted operational parameters and/or may cause the insulin pump to adjust operation of the insulin pump (e.g., may adjust timing for delivering insulin and/or an amount of insulin delivered).

An insulin pump system is provided herein that may be worn by a user and may be in wireless communication with a first device. The insulin pump system may include a pump which pumps insulin into a user based on first pump parameters, memory configured to store computer-executable instructions, and at least one computer processor associated with the pump, the at least one computer processor may access memory and execute the computer-executable instructions to: determine a computational operation is necessary to generate second pump parameters for operating the pump, determine, based on the first device having greater processing power than the at least one computer processor associated with the pump, that the first device is to perform the computational operation, send instructions to the first device to perform the computational operation on the first device to generate the second pump parameters, receive, after sending instructions to the first device, the second pump parameters from the first device, the second pump parameters different than the first pump parameters, and adjust, automatically, operation of the pump based on the second pump parameters upon receiving the second pump parameters.

The at least one computer processor of in the insulin pump system may further access memory and execute the computer executable instructions to send pump data corresponding to the computational operation to the first device and the second pump parameters may be generated by the first device based at least in part on the pump data. The first device may be a mobile phone and the mobile phone may have a greater memory and/or greater processing speed than the insulin pump system. The at least one computer processor of the insulin pump system may access memory and execute the computer executable instructions to send authentication data to the first device and/or may determine to generate second pump parameters based on sensor data and/or user input data.

The at least one computer processor of the insulin pump system may access memory and execute the computer executable instructions to determine the first device corresponds to the computational operation. The determination that the first device corresponds to the computational operation may be based on a table comprising a plurality of computational operations and associated devices for executing a respective one of the plurality of computational operations. The insulin pump system may further include second computer executable instructions designed to be executed by at least one computer processor on the first device to cause the first device to execute the computational operation to generate the second pump parameters. The pump may include an adhesive patch and may be a patch pump. The pump may include a battery and the first device may be a charger device for charging the battery.

Yet another insulin pump system is provided herein that may be worn by a user and may be in wireless communication with a first device. The insulin pump system may include a pump which pumps insulin into a user based on first pump parameters, memory designed to store computer-executable instructions, and at least one computer processor designed to access memory and execute the computer-executable instructions to: determine a computational operation is necessary to generate second pump parameters for operating the pump, determine that a second device is to perform the computational operation, send instructions to the first device to instruct the second device to perform the computational operation on the second device to generate the second pump parameters, receive, after sending instructions to the first device, the second pump parameters from the first device, the second pump parameters generated by the second device and different than the first pump parameters, and adjust, automatically, operation of the pump based on the second pump parameters upon receiving the second pump parameters.

The at least one computer processor of the insulin pump system may access memory and execute the computer executable instructions to send pump data corresponding to the computational operation to the first device and the second pump parameters may be generated by the second device based at least in part on the pump data. The first device may be a mobile phone and the second device may be a server and the first device and the at least one computer processor may be associated with the pump may communicate via a first network type and the first device and the second device may communicate via a second network type different than the first network type. The at least one computer processor of the insulin pump system may further access memory and execute the computer executable instructions to send authentication data to the first device.

The at least one computer processor of the insulin pump system may further access memory and execute the computer executable instructions to determine to generate second pump parameters based on sensor data and/or user input data. The at least one computer processor of the insulin pump system may further access memory and execute the computer executable instructions to determine the second device corresponds to the computational operation. The determination that the second device corresponds to the computational operation may be based on a table comprising a plurality of computational operations and associated devices for executing a respective one of the plurality of computational operations.

The insulin pump system may further include second computer executable instructions to be executed by at least one computer processor on the first device to cause the first device to send second instructions to the second device to cause the second device to execute the computational operation to generate the second pump parameters. The insulin pump system may further include third computer executable instructions to be executed by at least one computer processor on the second device to cause the second device to execute the computational operation to generate the second pump parameters. The pump may further include a battery, and the first device may be a charger device which may charge the battery. The pump may include an adhesive patch and may be a patch pump.

The foregoing summary is illustrative only and is not intended to be in any way limiting. In addition to the illustrative aspects, embodiments, and features described above, further aspects, embodiments, and features will become apparent by reference to the following drawings and the detailed description.

### BRIEF DESCRIPTION OF THE DRAWINGS

FIG. 1 illustrates an exemplary insulin pump system including a wearable pump and periphery and remote devices, in accordance with some aspects.

FIG. 2 illustrates an exemplary data flow depicting data exchanged between various components of the insulin pump system.

FIG. 3 illustrates an exemplary process flow for the insulin pump system for delegating an operation to a periphery or remote device.

FIG. 4 illustrates an exemplary process flow for the insulin pump system for executing an operation at a periphery or remote device.

FIG. 5 illustrates an exemplary insulin pump system including a patch pump and periphery and remote devices.

FIG. 6 illustrates a schematic block diagram of a pump in accordance with one or more exemplary embodiments of the disclosure.

The foregoing and other features of the present invention will become apparent from the following description and appended claims, taken in conjunction with the accompanying drawings. Understanding that these drawings depict only several embodiments in accordance with the disclosure and are, therefore, not to be considered limiting of its scope, the disclosure will be described with additional specificity and detail through use of the accompanying drawings.

### DETAILED DESCRIPTION

The present disclosure is directed to systems and methods for an insulin pump system including an insulin pump in communication with one or more periphery and/or remote devices. For example an insulin pump may be used by and worn by a user. The insulin pump may periodically deliver insulin to the user based on certain pump parameters (e.g., settings, rules, and/or constraints). The insulin pump may include a processor for operating the pump to achieve desired insulin delivery. Certain computations, models, algorithms, and/or operations may be needed to determine appropriate operation and/or adjustment of the pump. The insulin pump may instruct a periphery device, such as a mobile phone or smart watch, and/or a remote device, such as a server or other remote computing device, to perform such computations, models, algorithms, and/or operations, and send data, outputs, and/or results to the insulin pump for making such adjustments or other changes to the operation of the pump.

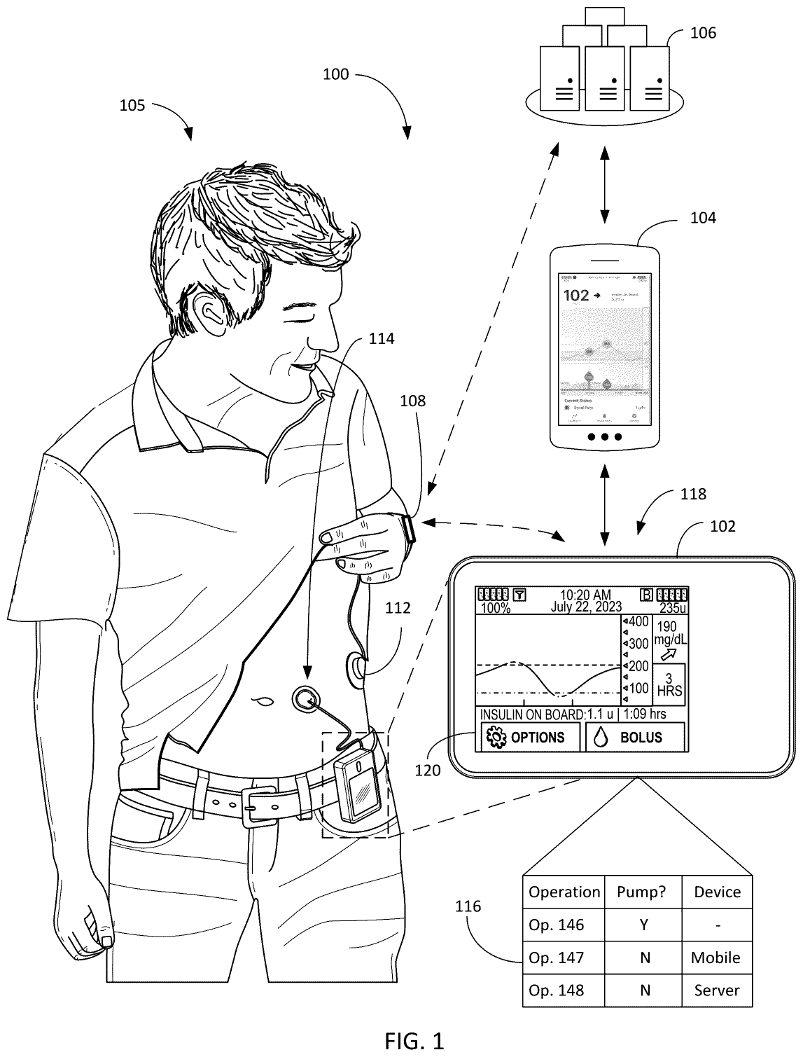

Referring now to FIG. 1, an insulin pump system including a wearable pump and periphery and remote devices is illustrated. Insulin pump system 100 may include insulin pump 102, mobile device 104, remote device 106, and/or smart device 108. Insulin pump system 100 may communicate with other devices such as glucose monitor 112 and/or other computing devices such as a healthcare provider device. Insulin pump system 100 may include greater or fewer devices than those illustrated in FIG. 1 and/or one or more device in FIG. 1 may be optional.

Insulin pump 102 may include pump housing 118 which may include a display and may house a pump designed to pump insulin into a user (e.g., user 105). For example, insulin pump 102 may be connected to delivery component 114 which may include tubing and/or a delivery patch having a needle or cannula for delivering insulin into a subcutaneous area of the user. Insulin pump 102 may include a compartment for holding a volume of insulin and may selectively deliver an amount of insulin (e.g., a bolus) to the user via the pump and delivery component 114.

Insulin pump 102 may, in one example, be the same as or similar to a t:slim X2™ insulin pump available from Tandem Diabetes Care, Inc. of San Diego, California and/or the insulin pump described in U.S. Patent App. Pub. No. 2014/0276423, published on Sep. 18, 2014 and assigned to Tandem Diabetes Care, Inc., hereby incorporated by reference in its entirety. However, it is understood that insulin pump 102 may be any type of insulin pump for delivering insulin to the user and having the functionality described herein. Insulin pump 102 may include one or more processors and memory as well as a communication chip for communicating with one or more periphery devices. For example, insulin pump 102 may communicate with mobile device 104 and/or smart device 108 via a suitable wireless connection (e.g., Bluetooth, Bluetooth Low Energy (BLE), Wi-Fi Direct, any other near field communication protocol, and/or the like). Insulin pump 102 may optionally communicate directly with remote device 106, glucose monitoring sensor 112 and/or any other devices.

As shown in FIG. 1, insulin pump 102 may be an extracorporeal and may be worn by the user. For example, insulin pump 102 may be worn on the user's belt or waist band. In another example, insulin pump 102 may be worn on a user's arm (e.g., using an arm band). The user may also have one or more periphery devices nearby. For example, the user may hold mobile device 104 in their pocket or personal bag (e.g., backpack or purse) and/or may wear smart device 108 (e.g., on their wrist). Due to the proximity of the periphery devices to insulin pump 102, insulin pump 102 may use short range communication technology (e.g., Wi-Fi, cellular network, Bluetooth, Bluetooth Low Energy (BLE), Wi-Fi Direct, near field communication protocol, and the like) for communication with the periphery devices.

Mobile device 104 may be any suitable mobile device such as a mobile phone (e.g., smart phone), tablet, laptop, wearable, charging device, smart device, and/or smart sensor. Mobile device 104 may include a computer processor, memory, and/or a communication unit which may facilitate communication between mobile device and insulin pump 102, remote device 106, smart device 108, and/or any other devices (e.g., healthcare provider device, smart sensors such as glucose monitoring sensor 112, and/or any other devices).

Mobile device 104 may communicate with any devices in insulin pump system 100 and any other devices via any suitable wired or wireless system (e.g., Wi-Fi, cellular network, Bluetooth, Bluetooth Low Energy (BLE), near field communication protocol, etc.). Mobile device 104 may support communication with various devices over different communication networks. For example, mobile device 104 may communicate with insulin pump 102 via one network type (e.g., a near field communication network or Bluetooth) and may communicate with remote device 106 via a different network type (e.g., cellular and/or Wi-Fi protocols).

Remote device 106 may be any computing device having a processor, memory and a communication unit. For example, remote device 106 may be one or more servers, datastores, or the like. Remote device 106 may communicate with any devices in insulin pump system 100 and any other devices via any well-known wired or wireless system (e.g., Wi-Fi, cellular network, Bluetooth, Bluetooth Low Energy (BLE), near field communication protocol, etc.).

Insulin pump 102, mobile device, 104, remote device 106, and/or smart device 108 may be in communication with glucose monitor 112, which may be any suitable continuous glucose monitoring sensor such as the DexCom G7 Continuous Glucose Monitoring (CGM) system available from DexCom, Inc. of San Diego, California and/or other sensor device. For example, insulin pump 102, mobile device, 104, remote device 104, and/or smart device 108 may receive glucose measurements and/or other health related information from glucose monitor 112 and may share such information with other devices in insulin pump system 100.

Insulin pump 102 may be used by the user to selectively deliver an amount of insulin (e.g., a bolus) to regulate the user's blood glucose levels. Insulin pump 102 may be programmed to deliver insulin based on certain rules and/or constraints (e.g., at certain times and/or volumes). Additionally, or alternatively, the insulin pump may deliver insulin based on input data received and/or determined (e.g., via user input and/or sensor data).

Insulin pump 102 may, for example, receive a blood glucose measurement reading generated by a sensor (e.g., glucose monitor 112). Insulin pump 102 may analyze the measurement and/or other glucose and/or biometric data (e.g., heart rate) and may determine whether or not to deliver insulin to a user and/or how much insulin to deliver. For example, if the measurement and/or data achieves a certain threshold value, insulin pump 102 may cause a set amount of insulin to be delivered to the user.

Additionally or alternatively, insulin pump 102 may deliver insulin to a user based on user input. For example, display 120 may be a touch display and a user may instruct insulin pump to deliver a certain amount of insulin and/or may input other information such as carbohydrate information and insulin pump may determine how much insulin to deliver based on this information.

Complex operations, calculations, policies and/or analysis have been developed to determine when and/or how much insulin to deliver to a user. For example, trends may be identified from input data such as blood glucose measurements and/or other biometric data (e.g., heart rate) and/or predictions may be made about future blood glucose measurements and/or estimates for an amount of insulin needed in certain scenarios. In one example a model predictive controller may determine pump parameters for adjusting pump operation.

While operations, tasks, calculations and/or analysis may improve dosing and management of blood glucose levels, such operations, calculations and/or analysis may include sophisticated algorithms, vast amounts of data, and/or complex operations and thus may require a significant amount of computer processing capability and/or demand a significant amount of processing power and may require a relatively large amount of energy to compute. Insulin pump 102 may be limited in processing power and/or speed and memory or other hardware as insulin pump 102 is limited in size and weight. Mobile device 104, smart device 108 and/or processor 106 may include more powerful and/or faster processors, a greater amount of memory, and even hardware (e.g., communication technologies) and/or software not available on insulin pump 102.

To perform operations, tasks, calculations and/or analysis not capable of being performed on insulin pump 102 and/or capable of being performed on insulin pump 102 but requiring too much processing power or energy (e.g., battery power), such operations, tasks, calculations and/or analysis may be delegated to another device (e.g., mobile device 104, smart device 108, and/or server 106). Alternatively, or additionally, insulin pump 102 may instruct another device in insulin pump system 100 to perform a task (e.g., present a video on display, play an alarm, send a message to a different computing device, and the like).

As shown in FIG. 1, insulin pump 102 may instruct mobile device 104 to execute a certain operation and/or to cause remote device 106 to execute a certain operation. Insulin pump 102 may optionally send data (e.g., pump data) or other information to mobile device 104, which may be relevant to the requested operation. Alternatively, or additionally, insulin pump 102 may send such instructions to smart device 108.

To determine whether the operations, tasks, calculations and/or analysis or the like should be performed locally on insulin pump 102 and/or by another device in insulin pump system 100, insulin pump 102 may associate certain operations with one or more devices in insulin pump system 100. In one example, insulin pump 102 may include table 116 which may indicate whether insulin pump 102 should perform such operation, task, calculation and/or analysis.

Table 116 may include several possible operations that may be performed and may associate a device with each operation, designating such device for performing the operation. For example, table 116 may indicate that an operation labeled operation 146 should be performed or executed by insulin pump 102, the operation labeled operation 147 should be performed by mobile device 104 and the operation labeled operation 148 should be performed by remote device 106. Insulin pump 102 may be programmed to send instructions to mobile device 104 to instruct mobile device 104 to perform an operation and/or send instructions to mobile device 104 to instruct mobile device 104 to instruct remote device 106 to perform the operation.

Based on table 116, insulin pump may determine which device to delegate such operations, tasks, calculations and/or analysis. Mobile device 104, remote device 106, and/or smart device 108 may perform the desired operation, task, calculation, analysis, or the like, and may send information back to insulin pump 102 (e.g., either directly or indirectly through mobile device 104 and/or smart device 108) which may include output data, results data, and/or any other information relating to the operation, task, calculation and/or analysis. In one example, Mobile device 104, remove device 106, and/or smart device 108 may send insulin pump 102 pump parameters for operating the pump. For example, insulin pump 102 may receive a table with policies and/or other information for operating insulin pump 102.

Insulin pump 102 may receive the information from mobile device 104, remote device 106, and/or smart device 108 and may, for example, determine pump parameters such adjusting operation of the pump and/or may determine updated or adjusted pump parameters for adjusting operation of the pump. Insulin pump 102 may automatically adjust operation of the pump upon receiving such pump parameters. Insulin pump 102 may present graphics and/or audio based on the information insulin pump 102 received from mobile device 104, remote device 106, and/or smart device 108.

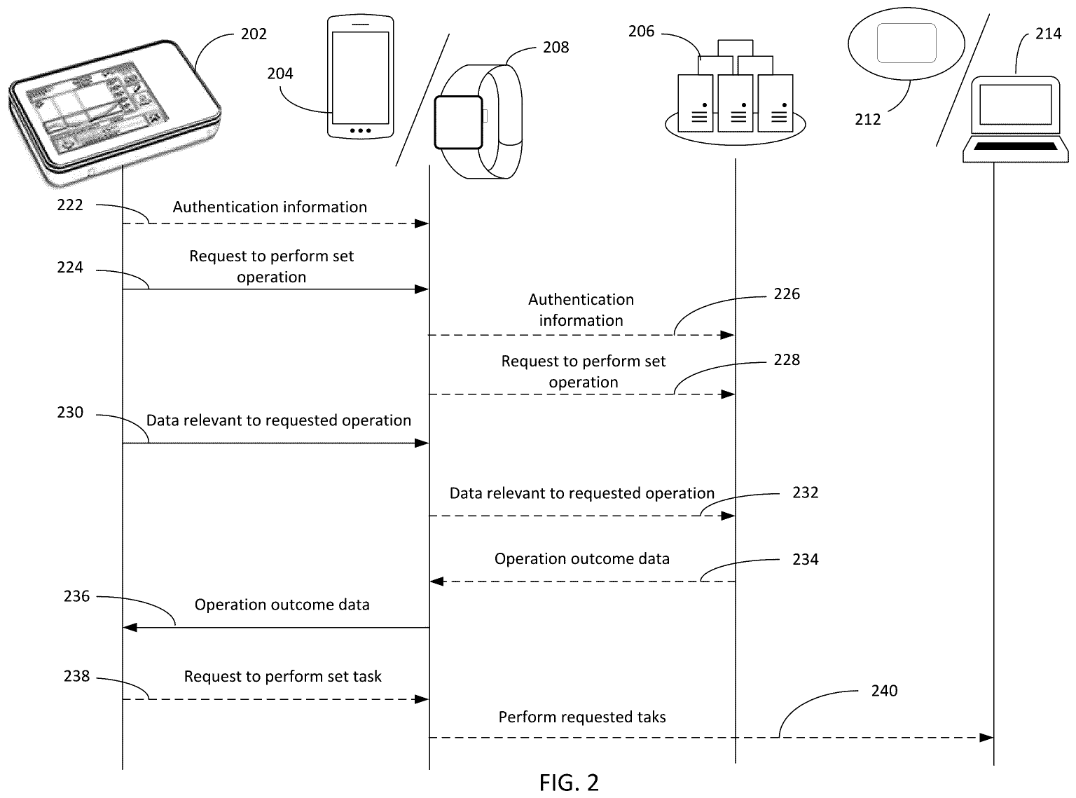

Referring now to FIG. 2, an exemplary data flow depicting data exchanged between various components of the insulin pump system and components outside of insulin pump system is illustrated. Specifically, FIG. 2 depicts data exchanges between insulin pump 202, mobile device 204, smart device 208, remote device 206, sensor device 212 and/or computing device 214. It is understood that one or more steps in FIG. 2 may be optional and/or additional or fewer steps than those shown in FIG. 2 may be performed.

Insulin pump 202, mobile device 204, smart device 208, and/or remote device 206 may be the same as or similar to insulin pump 102, mobile device 104, smart device 208, and/or remote device 206 of FIG. 1. Smart sensor 212 may be the same as or similar to glucose monitor 112 or may be any other sensor device (e.g., smart sensor). Computing device 114 may be any suitable computing device with a processor and a communication chip (e.g., mobile phone, smart phone, laptop, tablet, desktop, server, etc.). Smart sensor 212 and/or computing device 214 may be outside of the insulin pump system but may communicate with one or more components of the insulin pump system.

To initiate the data flow set forth in FIG. 2, at optional step 222, insulin pump 202 may send authentication information to mobile device 204 and/or smart device 208 to authenticate insulin pump 202. This may be performed the first time insulin pump 202 communicates with mobile device 204 and/or smart device 208 and/or may be included in every message sent from insulin pump 202. Authentication information may indicate the that insulin pump 202 is from a certain company, may include model information, may include user information, may include an authentication code and/or any other relevant information.

At step 224, insulin pump 202 may send a request or instructions to perform a set operation to mobile device 204 and/or smart device 208. The term operation is understood to include any operation, calculation, task, and/or the like. For example, the insulin pump may determine that a certain operation should be performed by a periphery device or a remote device. At optional step 226 mobile device 204 and/or smart device 208 may send authentication information and at optional step 228 may send the request to perform the set operation to remote device 206.

Similar to step 222, mobile device 204 and/or smart device 208 may send authentication information with each message and/or may send authentication with only the first message to remote device 206 such that remote device 206 may authenticate insulin pump 202. Insulin pump 202 may also instruct mobile device 204 and/or smart device 208 that the operation is to be performed by remote device 206. Alternatively, mobile device 204 and/or smart device 208 may make this determination.

At step 230, insulin pump 202 may send data relevant to the requested operation to mobile device and/or smart device 208. For example, certain data may be used in performing the operation and may be provided by insulin pump 202. In one example, such data may include historic data regarding insulin delivery, biometric data, data input from a user (e.g., using the touch display on insulin pump 202), and/or data received from a smart sensor (e.g., smart sensor 212). At optional set 232 mobile device 204 and/or smart device 208 may send such relevant data to remote device 206 if remote device 206 is to perform the operation. Mobile device 204 and/or smart device 208 may send different and/or additional data than that provided from pump 202. Alternatively, mobile device 204, smart device 208, and/or remote device 206 may determine or otherwise obtain relevant data that may be used in performing the operation.

If remote device 206 is instructed to perform the operation at step 228, then at optional step 234, remote device 206 may send operation outcome or output data generated by performing the requested operation to mobile device 204 and/or smart device 208. At step 236 mobile device 104 and/or smart device 108 may send such data to insulin pump 202. Alternatively, if mobile device 204 and/or smart device 208 perform the operation, then at step 236 operation outcome data may be sent from mobile device 204 and/or smart device 208 to insulin pump 202.

Operational outcome data (e.g., output data) may be generated by performing the operation, which may optionally include using the relevant data provided by insulin pump 202. Operation outcome data may include for example, pump parameters that may be used by insulin pump 202 to change the operation of the pump, pump schedule information, insulin dosing information, operational instructions for hardware of insulin pump 202 such as battery and/or pump operation instructions, display and/or audio information, and/or any other relevant information for operating insulin pump 202.

At optional step 238, insulin pump 202 may request that mobile device 204 and/or smart device 208 perform a set task. For example, insulin pump 202 may be limited in hardware and/or mobile device 204 and/or smart device 208 may have different hardware, capability, and/or functionality than insulin pump 202. In one example, insulin pump 202 may not have cellular connectivity and insulin pump 202 send instructions to mobile device 204 and/or smart device 208 to contact mobile device 214 with a message and/or information relevant to operation of insulin pump 202, for example. In another example, mobile device 204 may be in communication with smart sensor 212 and insulin pump 202 may request that mobile device 204 obtain data from smart sensor 212 and share such data with insulin pump 202.

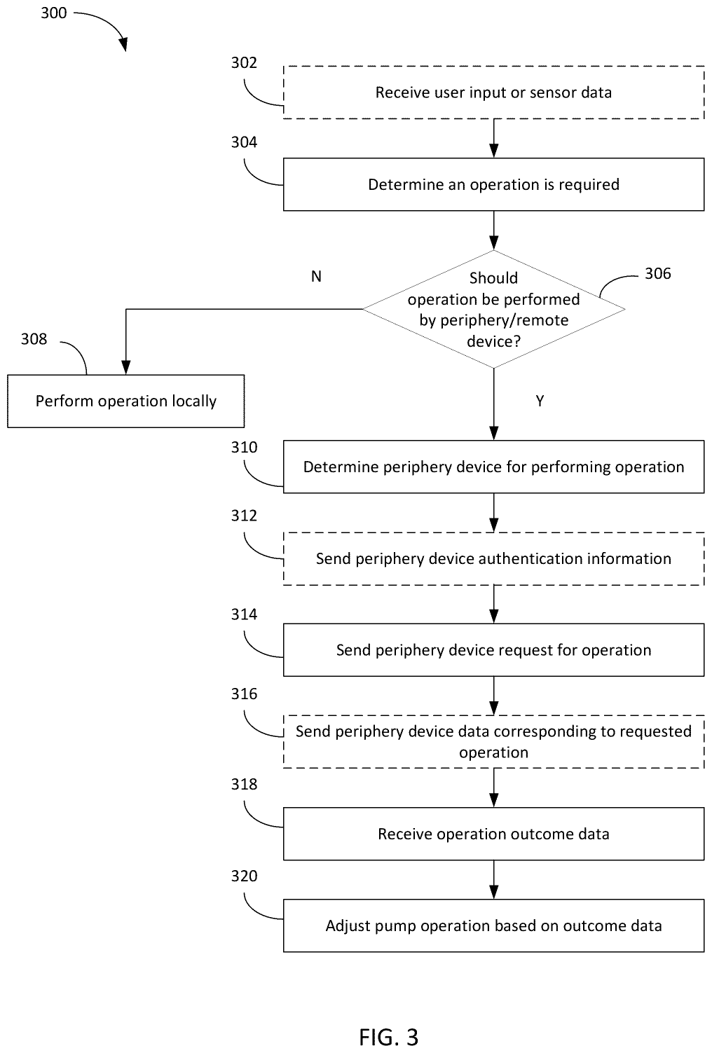

Referring now to FIG. 3, an exemplary process flow for the insulin pump system for delegating an operation to a periphery or remote device is illustrated. Some or all of the blocks of the process flow in FIG. 3 may be performed in a distributed manner across any number of devices (e.g., insulin pump, periphery devices, remote devices, computing devices and/or servers). Some or all of the operations of the process flow may be optional and may be performed in a different order.

To initiate process flow 300, at optional block 302, computer-executable instructions stored on a memory of a device, such as an insulin pump, may be executed to receive and/or determine user input data and/or sensor data. For example, user input data may be received from a touchscreen of the insulin pump and may include dosing and/or carbohydrate intake information. Sensor data may come from a smart sensor and/or may include information such as blood glucose measurements, for example.

At block 304, computer-executable instructions stored on a memory of a device, such as an insulin pump, may be executed to determine that an operation is required. Operations may be any computational operation and/or may be any operation, computation, calculation, tasks, analysis and/or the like. For example, certain operations may be performed periodically or based on a schedule, operations may be triggered by other events (e.g., upon a certain threshold being satisfied such as a blood glucose threshold and/or upon receiving user input).

At decision 306, computer-executable instructions stored on a memory of a device, such as an insulin pump, may be executed to determine whether the operation should be performed by the insulin pump or by a periphery or remote device. To make this decision a table or other information associating certain operations with periphery and/or remote devices may be consulted. For example, each operation may be labeled and may have an associated device designated for performing the operation.

If the operation should be performed by the insulin pump itself, at block 308, computer-executable instructions stored on a memory of a device, such as an insulin pump, may be executed to perform the operation locally on the insulin pump. Alternatively, if the operation should be performed by a periphery device and/or a remote device, then at block 310 computer-executable instructions stored on a memory of a device, such as an insulin pump, may be executed to determine a periphery device or remote device for performing the operation. This may be done by consulting the table or by determining a periphery device and/or remote device associated with insulin pump.

At optional block 312, computer-executable instructions stored on a memory of a device, such as an insulin pump, may be executed to send to send a periphery device authentication information. For example, if a periphery device is determined at block 310, then this may be the same periphery device determined at block 310. Alternatively, if a remote device is determined at decision 306, then the periphery device may be used by the insulin pump to relay messages to the remote device.

At block 314, computer-executable instructions stored on a memory of a device, such as an insulin pump, may be executed to send the periphery device the request to perform the operation. The request may be for the periphery device to perform the operation or for the periphery device to instruct a remote device to perform the operation. For example, the periphery device and/or remote device may store and/or have access to data that the insulin pump does not have and/or have access to. For example, due to hardware (e.g., memory) constraints, the insulin pump may not store such data and instead the data may be collected and/or stored externally. In such circumstances, the insulin pump may request that such periphery and/or remote device perform the operation and apply and/or consider the additional data. In this manner the additional data may be considered and/or applied when performing the operation. It is understood that if the periphery and/or remote device is unavailable, then the insulin pump may optionally perform the operation without the additional data.

In one example, the insulin pump may be in communication with a mobile device (e.g., mobile phone). The mobile device may collect data that may not be directly accessible or available to the insulin pump (e.g., biometric data such as an amount of steps taken by a user and/or heart rate data). As such information may be informative for making a better glucose prediction, for example, the insulin pump may request that the mobile device perform the operation (e.g., glucose prediction) using the additional data (e.g., the biometric data). In another example, a remote device (e.g., server) may maintain a history of a user's glucose levels, insulin doses, and/or any other relevant information (e.g., a set of calculated parameters based on the glucose levels and/or insulin doses). The insulin device may request that the remote device perform an operation (e.g., determine a glucose prediction and/or insulin dose calculation) using the additional data stored on the remote device.

At optional block 316, computer-executable instructions stored on a memory of a device, such as an insulin pump, may be executed to send the periphery device data relevant to the requested operation. This information may be used by the periphery device and/or remote device in performing the operation.

At block 318, computer-executable instructions stored on a memory of a device, such as an insulin pump, may be executed to determine and/or receive outcome data from the operation performed by the periphery device and/or the remote device. The outcome data may be received from the periphery device. At block 320, computer-executable instructions stored on a memory of a device, such as an insulin pump, may be executed to adjust pump operation based on the outcome data.

The outcome data may be used by the insulin pump to determine and/or adjust when the insulin is delivered and dosing (e.g., volume) insulin delivered by the insulin pump. In one example, the outcome data may be adjusted pump parameters for operating the pump. For example, the insulin pump may receive adjusted pump parameters from the periphery device and may automatically adjust operation of the pump based on the adjusted pump parameters.

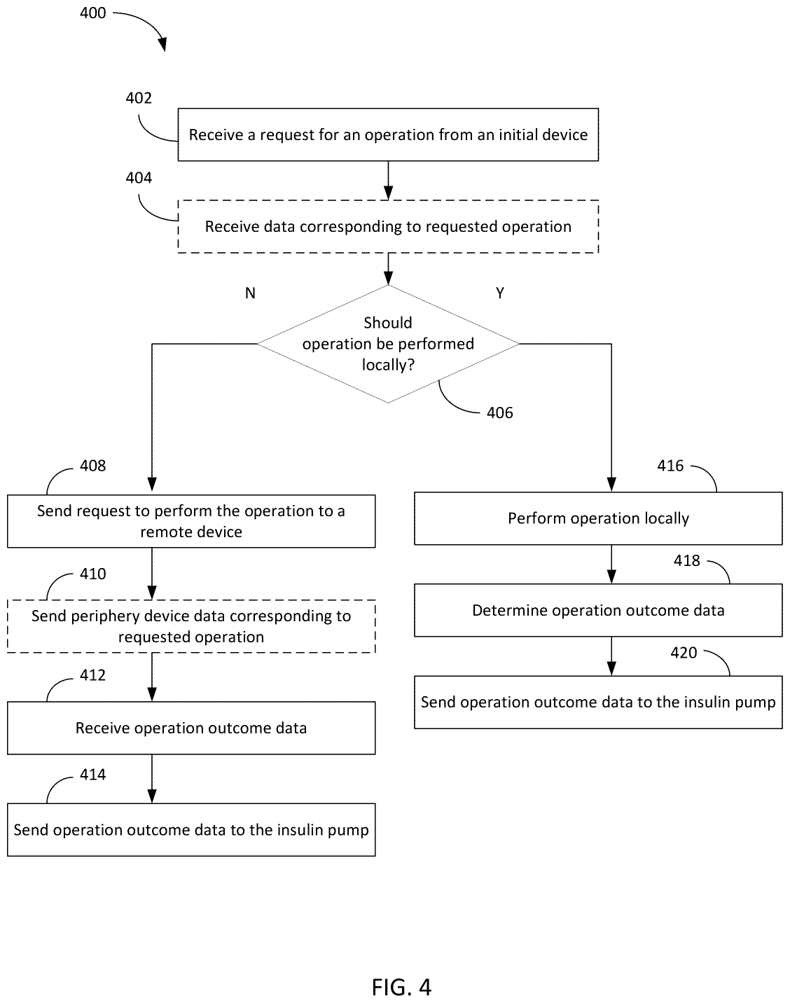

Referring now to FIG. 4, an exemplary process flow for executing an operation at a periphery or remote device is illustrated. Some or all of the blocks of the process flow in FIG. 4 may be performed in a distributed manner across any number of devices (e.g., periphery devices, remote devices, insulin pump, computing devices and/or servers). Some or all of the operations of the process flow may be optional and may be performed in a different order.

To initiate process flow 400, at block 402, computer-executable instructions stored on a memory of a device, such as a periphery device (e.g., mobile device and/or smart device) or a remote device, may be executed to receive and/or determine a request for an operation from an initial device. The initial device may either be the insulin pump or a periphery device, in the case of the remote device.

At optional block 404, computer-executable instructions stored on a memory of a device, such as a periphery device or a remote device, may be executed to receive data corresponding to the requested operation. This data may be the same or similar to the data in block 316 of FIG. 3. It is understood that authentication information may optionally be received as well.

At decision 406, computer-executable instructions stored on a memory of a device, such as a periphery device or a remote device, may be executed to determine whether the operation should performed locally (e.g., on the device that received the request at block 402). If the device that received the request at block 402 is a periphery device and the operation is to be performed by the remote device, then the operation should not be performed locally and at block 408 computer-executable instructions stored on a memory of a device, such as a periphery device, may be executed to send the request for to perform the operation to the remote device.

At optional block 410, computer-executable instructions stored on a memory of a device, such as a periphery device, may be executed to also send the remote device the data corresponding to the requested operation. At block 412, computer-executable instructions stored on a memory of a device, such as a periphery device, may be executed to receive the outcome data from the remote device. The outcome data may be the same as or similar to the outcome data received at block 318 of FIG. 3. At block 414, computer-executable instructions stored on a memory of a device, such as a periphery device, may be executed to send the outcome data to the insulin pump.

If instead at decision 406, the device that received the request at block 402 is the remote device and the operation is to be performed by the remote device or is the periphery device and the operation is to be performed by the periphery device, then the operation should be performed locally. In this case at block 408, computer-executable instructions stored on a memory of a device, such as a periphery device or a remote device, may be executed to perform the operation locally (e.g., on the same device that received the request at block 402).

At block 418, computer-executable instructions stored on a memory of a device, such as a periphery device or a remote device, may be executed to determine the outcome data. At block 420, computer-executable instructions stored on a memory of a device, such as a periphery device or a remote device, may be executed to send the outcome data to the insulin pump. For example, if the operation data is determined by the periphery device, then the periphery device may send the operation data directly to the insulin pump. Alternatively, if the remote device is the device that determines the outcome data, then the outcome data may be sent to the insulin pump by the remote device via the periphery device as a relay.

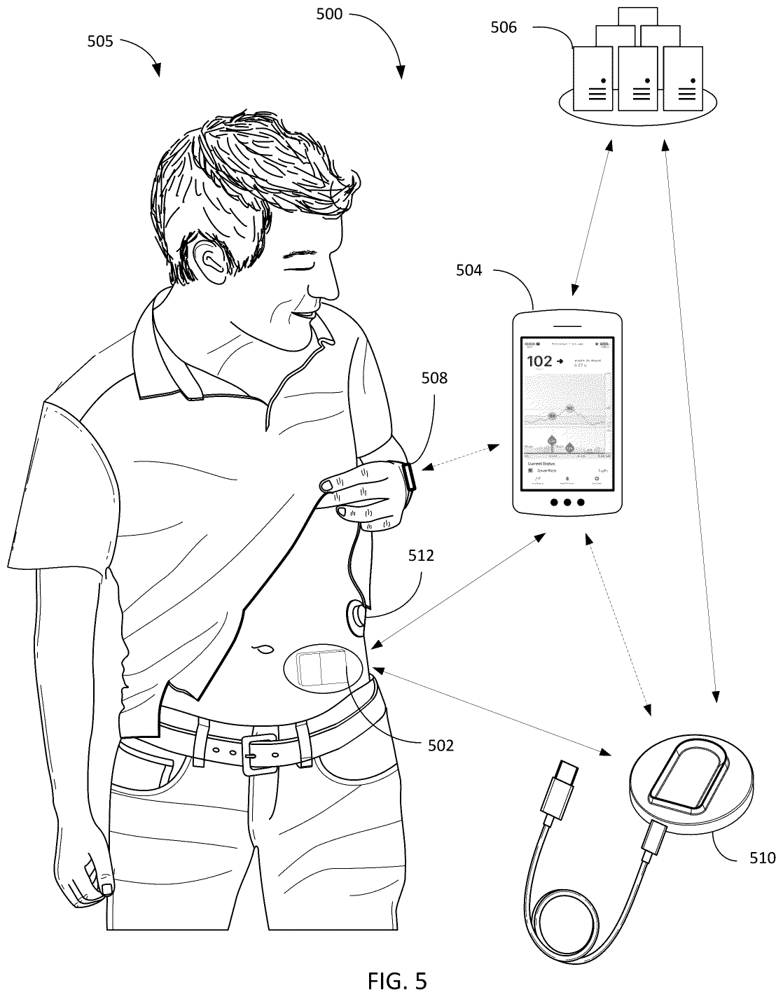

Referring now to FIG. 5, an exemplary insulin pump system including a patch pump and periphery and remote devices is illustrated. Specifically, insulin pump system 500 may include patch pump 502, mobile device 504, smart device 508, remote device 506 and/or charger 510. Mobile device 504, smart device 508, remote device 506, and/or glucose monitoring device 512 may be the same as or similar to mobile device 104, smart device 108, remote device 106, and/or glucose monitoring device 512 of FIG. 1. Insulin pump system 500 may communicate with other devices such as glucose monitor 112 and/or other computing devices such as a healthcare provider device. Insulin pump system 500 may include greater or fewer devices than those illustrated in FIG. 5 and/or one or more device in FIG. 5 may be optional.

Patch pump 502 may be any type of insulin pump that may include a pump housing connected to an adhesive patch for securing patch pump 502 to the user's skin (e.g. user 505). Patch pump 502 may include a container for storing a volume of insulin, a pump and a needle or cannula through which insulin may be delivered subcutaneously to the user. In one example, patch pump 502 may be the same as or similar to the patch pump described in U.S. Pat. No. 11,529,460 issued on Dec. 20, 2022, and assigned to AMF Medical SA, hereby incorporated by reference in its entirety.

Patch pump 502 may further include one or more processors, communications units, and/or rechargeable batteries in the housing. For example, patch pump 502 may communicate with charger 510, smart device 508, mobile device 504, remote device 506, and/or any other devices (e.g., glucose monitoring device 512) via any suitable wired or wireless communication technology (e.g., Bluetooth, Bluetooth Low Energy (BLE), Wi-Fi Direct, cellular, and/or the like). As shown in FIG. 5, patch pump 502 may communicate directly with mobile device 504 and/or charging device 510 (e.g., via a near field technology protocol, Bluetooth, BLE, Wi-Fi Direct, etc.).

The rechargeable battery of patch pump 502 may be charged by charging device 510. Charging device 510 may facilitate wireless charging via one or more induction coils. Patch pump 502 may similarly have one or more induction coils and the rechargeable battery may be charged when patch pump 502 is positioned onto charging device 510. Charger 510 may further including one or more processors and/or a communication unit. For example, charger 510 may communication with patch pump 502, smart device 508, mobile device 504, remote device 506, and/or any other devices (e.g., glucose monitoring device 512) via any suitable wired or wireless communication technology (e.g., Bluetooth, Bluetooth Low Energy (BLE), Wi-Fi Direct, cellular, and/or the like).

As shown in FIG. 5, charging device 510 may communicate directly with patch pump 502 and/or remote device 506. In one example, charging device 510 may communicate with patch pump 502 (e.g., via a near field technology protocols, Bluetooth, Wi-Fi Direct, etc.) and may communicate with remote device 506 via cellular or Wi-Fi protocols for example. As is also shown in FIG. 504, mobile device 504 may communicate with charging device 510, smart device 508, patch pump 502, and/or remote device 506.

Patch pump 502, while different from insulin pump 102 of FIG. 1, may perform the same operations described with respect to insulin pump 102 in FIG. 1 and any other insulin pump described with respect to FIGS. 2-4. For example, patch pump 502 may perform the steps in FIG. 4 described with respect to insulin pump 102. In this manner, patch pump 502 may delegate certain operations to periphery devices and/or remote devices to be performed by a respective periphery device and/or remote device. With respect to patch pump 502, periphery devices may include charging device 510, mobile device 504, and/or smart device 508.

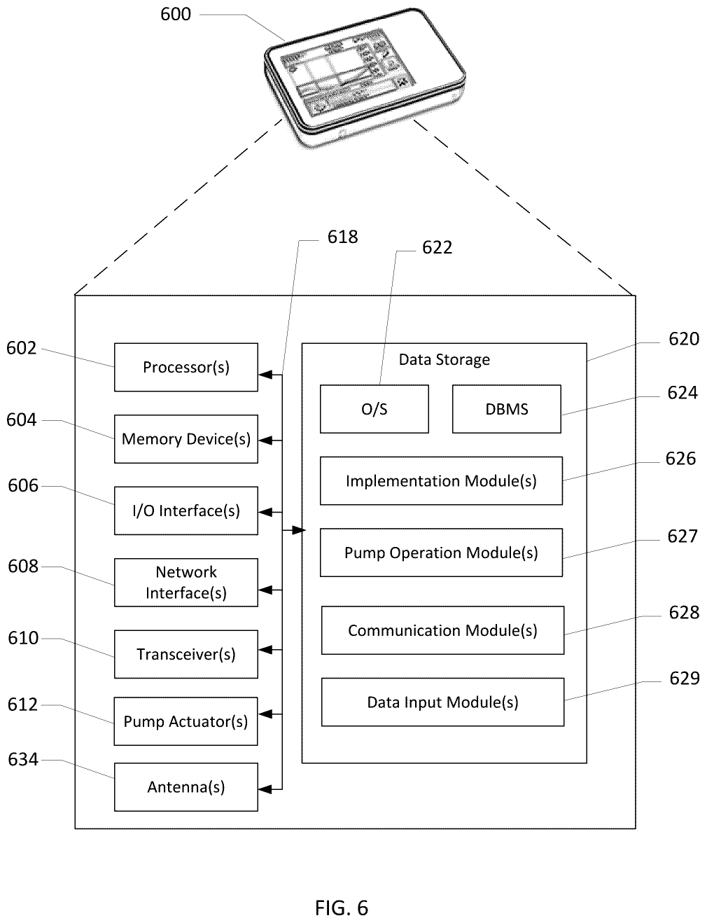

FIG. 6 is a schematic block diagram of illustrative insulin pump 600, which may be in communication with one or more periphery and/or remote devices, is illustrated. Insulin pump 600 may be the same or similar to insulin pump 102 of FIG. 1 or otherwise one or more of the insulin pumps or patch pumps of FIGS. 2-5. It is understood that an insulin pump 600 may alone or together with one or periphery or remote devices perform one or more of the operations of insulin pump 600.

Insulin pump 600 may be designed to communicate with one or more periphery devices, remote devices, smart devices, computing devices, servers, other systems, or the like. Insulin pump 600 may be designed to communicate via one or more networks. Such network(s) may include, but are not limited to, any one or more different types of communications networks such as, for example, near field communication networks, cable networks, public networks (e.g., the Internet), private networks (e.g., frame-relay networks), wireless networks, cellular networks, telephone networks (e.g., a public switched telephone network), or any other suitable private or public packet-switched or circuit-switched networks.

In an illustrative configuration, insulin pump 600 may include one or more processors 602, one or more memory devices 604 (also referred to herein as memory 604), one or more input/output (I/O) interface(s) 606, one or more network interface(s) 608, one or more transceiver(s) 610, one or more pump actuator(s) 612, one or more antenna(s) 634, and data storage 620. Insulin pump 600 may further include one or more bus(es) 618 that functionally couple various components of the insulin pump 600.

The bus(es) 618 may include at least one of a system bus, a memory bus, an address bus, or a message bus, and may permit exchange of information (e.g., data (including computer-executable code), signaling, etc.) between various components of the insulin pump 600. The bus(es) 618 may include, without limitation, a memory bus or a memory controller, a peripheral bus, an accelerated graphics port, and so forth. The bus(es) 618 may be associated with any suitable bus architecture including.

The memory 604 may include volatile memory (memory that maintains its state when supplied with power) such as random access memory (RAM) and/or non-volatile memory (memory that maintains its state even when not supplied with power) such as read-only memory (ROM), flash memory, ferroelectric RAM (FRAM), and so forth. Persistent data storage, as that term is used herein, may include non-volatile memory. In various implementations, the memory 604 may include multiple different types of memory such as various types of static random access memory (SRAM), various types of dynamic random access memory (DRAM), various types of unalterable ROM, and/or writeable variants of ROM such as electrically erasable programmable read-only memory (EEPROM), flash memory, and so forth.

The data storage 620 may include removable storage and/or non-removable storage including, but not limited to, magnetic storage, optical disk storage, and/or tape storage. The data storage 620 may provide non-volatile storage of computer-executable instructions and other data. The memory 604 and the data storage 620, removable and/or non-removable, are examples of computer-readable storage media (CRSM) as that term is used herein. The data storage 620 may store computer-executable code, instructions, or the like that may be loadable into the memory 604 and executable by the processor(s) 602 to cause the processor(s) 602 to perform or initiate various operations. The data storage 620 may additionally store data that may be copied to memory 604 for use by the processor(s) 602 during the execution of the computer-executable instructions. Moreover, output data generated as a result of execution of the computer-executable instructions by the processor(s) 602 may be stored initially in memory 604, and may ultimately be copied to data storage 620 for non-volatile storage.

The data storage 620 may store one or more operating systems (O/S) 622; one or more optional database management systems (DBMS) 624; and one or more program module(s), applications, engines, computer-executable code, scripts, or the like such as, for example, one or more implementation modules 626, one or more pump operation modules 627, one or more communication modules 628, and/or one or more data input modules 629. Some or all of these modules may be sub-modules. Any of the components depicted as being stored in data storage 620 may include any combination of software, firmware, and/or hardware. The software and/or firmware may include computer-executable code, instructions, or the like that may be loaded into the memory 604 for execution by one or more of the processor(s) 602. Any of the components depicted as being stored in data storage 620 may support functionality described in reference to correspondingly named components earlier in this disclosure.

Referring now to other illustrative components depicted as being stored in data storage 620, O/S 622 may be loaded from data storage 620 into memory 604 and may provide an interface between other application software executing on insulin pump 600 and hardware resources of the insulin pump 600. More specifically, O/S 622 may include a set of computer-executable instructions for managing hardware resources of insulin pump 600 and for providing common services to other application programs (e.g., managing memory allocation among various application programs). In certain example embodiments, O/S 622 may control execution of the other program module(s) for content rendering. O/S 622 may include any operating system now known or which may be developed in the future including, but not limited to, any server operating system, any mainframe operating system, or any other proprietary or non-proprietary operating system.

Optional DBMS 624 may be loaded into the memory 604 and may support functionality for accessing, retrieving, storing, and/or manipulating data stored in memory 604 and/or data stored in data storage 620. DBMS 624 may use any of a variety of database models (e.g., relational model, object model, etc.) and may support any of a variety of query languages. DBMS 624 may access data represented in one or more data schemas and stored in any suitable data repository including, but not limited to, databases (e.g., relational, object-oriented, etc.), file systems, flat files, distributed datastores in which data is stored on more than one node of a computer network, peer-to-peer network datastores, or the like.

I/O interface(s) 606 may facilitate the receipt of input information by insulin pump 600 from one or more I/O devices as well as the output of information from insulin pump 600 to the one or more I/O devices. The I/O devices may include any of a variety of components such as a touchscreen display; an audio output device for producing sound, such as a speaker; an audio capture device, such as a microphone; buttons and/or dials; and so forth. Any of these components may be integrated into insulin pump 600 or may be separate.

Insulin pump 600 may further include one or more network interface(s) 608 via which insulin pump 600 may communicate with any of a variety of other systems, platforms, networks, devices, and so forth. Network interface(s) 608 may enable communication, for example, with one or more wireless routers, one or more host servers, one or more web servers, and the like via one or more of networks.

Antenna(s) 634 may include any suitable type of antenna depending, for example, on the communications protocols used to transmit or receive signals via antenna(s) 634. Non-limiting examples of suitable antennas may include directional antennas, non-directional antennas, dipole antennas, folded dipole antennas, patch antennas, multiple-input multiple-output (MIMO) antennas, or the like. Antenna(s) 634 may be communicatively coupled to one or more transceivers 610 or radio components to which or from which signals may be transmitted or received. Antenna(s) 634 may include, without limitation, a cellular antenna for transmitting or receiving signals to/from a cellular network infrastructure, an antenna for transmitting or receiving Wi-Fi signals to/from an access point (AP), a Global Navigation Satellite System (GNSS) antenna for receiving GNSS signals from a GNSS satellite, a Bluetooth antenna for transmitting or receiving Bluetooth signals including BLE signals, a Near Field Communication (NFC) antenna for transmitting or receiving NFC signals, a 900 MHz antenna, and so forth.

Transceiver(s) 610 may include any suitable radio component(s) for, in cooperation with the antenna(s) 634, transmitting or receiving radio frequency (RF) signals in the bandwidth and/or channels corresponding to the communications protocols utilized by insulin pump 600 to communicate with other devices. Transceiver(s) 610 may include hardware, software, and/or firmware for modulating, transmitting, or receiving—potentially in cooperation with any of antenna(s) 634—communications signals according to any of the communications protocols discussed above including, but not limited to, one or more Wi-Fi and/or Wi-Fi direct protocols, as standardized by the IEEE 802.11 standards, one or more non-Wi-Fi protocols, or one or more cellular communications protocols or standards. Transceiver(s) 610 may further include hardware, firmware, or software for receiving GNSS signals. Transceiver(s) 610 may include any known receiver and baseband suitable for communicating via the communications protocols utilized by the insulin pump 600. The transceiver(s) 610 may further include a low noise amplifier (LNA), additional signal amplifiers, an analog-to-digital (A/D) converter, one or more buffers, a digital baseband, or the like.

Referring now to functionality supported by the various program module(s) depicted in FIG. 6, implementation module(s) 626 may include computer-executable instructions, code, or the like that responsive to execution by one or more of the processor(s) 602 may perform functions including, but not limited to, overseeing coordination and interaction between one or more modules and computer executable instructions in data storage 620, determining user actions, determining actions associated with user interactions, determining actions associated with user input, initiating commands locally or at periphery and/or remote devices, and the like.

Pump operation module(s) 627 may include computer-executable instructions, code, or the like that responsive to execution by one or more of the processor(s) 602 may perform functions including, but not limited to, overseeing, monitoring, and/or operating the pump and pump actuators for select delivery of a volume of insulin.

Communication module(s) 628 may include computer-executable instructions, code, or the like that responsive to execution by one or more of the processor(s) 602 may perform functions including, but not limited to, communicating with one or more devices, for example, via wired or wireless communication, communicating with mobile devices, smart devices, remote devices, charging devices, computing devices, smart sensors and/or any other devices.

The data input module(s) 629 may include computer-executable instructions, code, or the like that responsive to execution by one or more of the processor(s) 602 may perform functions including, but not limited to, determining overseeing, interpreting, managing, and/or analyzing input data such as input data received from a user via I/O interfaces 606, for example, or input data received from any other smart sensor, computing device, periphery device and/or remote device and the like.

Although specific embodiments of the disclosure have been described, one of ordinary skill in the art will recognize that numerous other modifications and alternative embodiments are within the scope of the disclosure. For example, any of the functionality and/or processing capabilities described with respect to a particular device or component may be performed by any other device or component. Further, while various illustrative implementations and architectures have been described in accordance with embodiments of the disclosure, one of ordinary skill in the art will appreciate that numerous other modifications to the illustrative implementations and architectures described herein are also within the scope of this disclosure.

Certain aspects of the disclosure are described above with reference to block and flow diagrams of systems, methods, apparatuses, and/or computer program products according to example embodiments. It will be understood that one or more blocks of the block diagrams and flow diagrams, and combinations of blocks in the block diagrams and the flow diagrams, respectively, may be implemented by execution of computer-executable program instructions. Likewise, some blocks of the block diagrams and flow diagrams may not necessarily need to be performed in the order presented, or may not necessarily need to be performed at all, according to some embodiments. Further, additional components and/or operations beyond those depicted in blocks of the block and/or flow diagrams may be present in certain embodiments.

Accordingly, blocks of the block diagrams and flow diagrams support combinations of means for performing the specified functions, combinations of elements or steps for performing the specified functions, and program instruction means for performing the specified functions. It will also be understood that each block of the block diagrams and flow diagrams, and combinations of blocks in the block diagrams and flow diagrams, may be implemented by special-purpose, hardware-based computer systems that perform the specified functions, elements or steps, or combinations of special-purpose hardware and computer instructions.

Program module(s), applications, or the like disclosed herein may include one or more software components, including, for example, software objects, methods, data structures, or the like. Each such software component may include computer-executable instructions that, responsive to execution, cause at least a portion of the functionality described herein (e.g., one or more operations of the illustrative methods described herein) to be performed.

A software component may be coded in any of a variety of programming languages. An illustrative programming language may be a lower-level programming language such as an assembly language associated with a particular hardware architecture and/or operating system platform. A software component comprising assembly language instructions may require conversion into executable machine code by an assembler prior to execution by the hardware architecture and/or platform.

Another example programming language may be a higher-level programming language that may be portable across multiple architectures. A software component comprising higher-level programming language instructions may require conversion to an intermediate representation by an interpreter or a compiler prior to execution.

Other examples of programming languages include, but are not limited to, a macro language, a shell or command language, a job control language, a script language, a database query or search language, or a report writing language. In one or more example embodiments, a software component comprising instructions in one of the foregoing examples of programming languages may be executed directly by an operating system or other software component without having to be first transformed into another form.

A software component may be stored as a file or other data storage construct. Software components of a similar type or functionally related may be stored together such as, for example, in a particular directory, folder, or library. Software components may be static (e.g., pre-established or fixed) or dynamic (e.g., created or modified at the time of execution).

Software components may invoke or be invoked by other software components through any of a wide variety of mechanisms. Invoked or invoking software components may comprise other custom-developed application software, operating system functionality (e.g., device drivers, data storage (e.g., file management) routines, other common routines, and services, etc.), or third-party software components (e.g., middleware, encryption, or other security software, database management software, file transfer or other network communication software, mathematical or statistical software, image processing software, and format translation software).

Software components associated with a particular solution or system may reside and be executed on a single platform or may be distributed across multiple platforms. The multiple platforms may be associated with more than one hardware vendor, underlying chip technology, or operating system. Furthermore, software components associated with a particular solution or system may be initially written in one or more programming languages, but may invoke software components written in another programming language.

Computer-executable program instructions may be loaded onto a special-purpose computer or other particular machine, a processor, or other programmable data processing apparatus to produce a particular machine, such that execution of the instructions on the computer, processor, or other programmable data processing apparatus causes one or more functions or operations specified in the flow diagrams to be performed. These computer program instructions may also be stored in a CRSM that upon execution may direct a computer or other programmable data processing apparatus to function in a particular manner, such that the instructions stored in the computer-readable storage medium produce an article of manufacture including instruction means that implement one or more functions or operations specified in the flow diagrams. The computer program instructions may also be loaded onto a computer or other programmable data processing apparatus to cause a series of operational elements or steps to be performed on the computer or other programmable apparatus to produce a computer-implemented process.

Additional types of CRSM that may be present in any of the devices described herein may include, but are not limited to, programmable random access memory (PRAM), SRAM, DRAM, RAM, ROM, electrically erasable programmable read-only memory (EEPROM), flash memory or other memory technology, compact disc read-only memory (CD-ROM), digital versatile disc (DVD) or other optical storage, magnetic cassettes, magnetic tape, magnetic disk storage or other magnetic storage devices, or any other medium which can be used to store the information and which can be accessed. Combinations of any of the above are also included within the scope of CRSM. Alternatively, computer-readable communication media (CRCM) may include computer-readable instructions, program module(s), or other data transmitted within a data signal, such as a carrier wave, or other transmission. However, as used herein, CRSM does not include CRCM.

Although embodiments have been described in language specific to structural features and/or methodological acts, it is to be understood that the disclosure is not necessarily limited to the specific features or acts described. Rather, the specific features and acts are disclosed as illustrative forms of implementing the embodiments. Conditional language, such as, among others, “can,” “could,” “might,” or “may,” unless specifically stated otherwise, or otherwise understood within the context as used, is generally intended to convey that certain embodiments could include, while other embodiments do not include, certain features, elements, and/or steps. Thus, such conditional language is not generally intended to imply that features, elements, and/or steps are in any way required for one or more embodiments or that one or more embodiments necessarily include logic for deciding, with or without user input or prompting, whether these features, elements, and/or steps are included or are to be performed in any particular embodiment.

It should be understood that any of the computer operations described herein above may be implemented at least in part as computer-readable instructions stored on a computer-readable memory. It will of course be understood that the embodiments described herein are illustrative, and components may be arranged, substituted, combined, and designed in a wide variety of different configurations, all of which are contemplated and fall within the scope of this disclosure.

The foregoing description of illustrative embodiments has been presented for purposes of illustration and of description. It is not intended to be exhaustive or limiting with respect to the precise form disclosed, and modifications and variations are possible in light of the above teachings or may be acquired from practice of the disclosed embodiments. It is intended that the scope of the invention be defined by the claims appended hereto and their equivalents.

What is claimed is:

1. An insulin pump system worn by a user and in wireless communication with a first device, the insulin pump system comprising:
a pump configured to pump insulin into the user based on first pump parameters;
memory configured to store computer-executable instructions; and
at least one computer processor associated with the pump, the at least one computer processor configured to access memory and execute the computer-executable instructions to:
determine a computational operation is necessary to generate second pump parameters for operating the pump;
determine, based on the first device having greater processing power than the at least one computer processor associated with the pump, that the first device is to perform the computational operation;
send instructions to the first device to perform the computational operation on the first device to generate the second pump parameters;
receive, after sending instructions to the first device, the second pump parameters from the first device, the second pump parameters different than the first pump parameters; and
adjust, automatically, operation of the pump based on the second pump parameters upon receiving the second pump parameters.

2. The insulin pump system of claim 1, wherein the at least one computer processor is further configured to access memory and execute the computer executable instructions to send pump data corresponding to the computational operation to the first device and the second pump parameters are generated by the first device based at least in part on the pump data.

3. The insulin pump system of claim 1, wherein the first device is a mobile phone and the mobile phone comprises greater memory and/or greater processing speed than the insulin pump system.

4. The insulin pump system of claim 1, wherein the at least one computer processor is further configured to access memory and execute the computer executable instructions to send authentication data to the first device.

5. The insulin pump system of claim 1, wherein the at least one computer processor is further configured to access memory and execute the computer executable instructions to determine to generate second pump parameters based on sensor data and/or user input data.

6. The insulin pump system of claim 1, wherein the at least one computer processor is further configured to access memory and execute the computer executable instructions to determine the first device corresponds to the computational operation.

7. The insulin pump system of claim 6, wherein the determination that the first device corresponds to the computational operation is based on a table comprising a plurality of computational operations and associated devices for executing a respective one of the plurality of computational operations.

8. The insulin pump system of claim 1, further comprising second computer executable instructions configured to be executed by at least one computer processor on the first device to cause the first device to execute the computational operation to generate the second pump parameters.

9. The insulin pump system of claim 1, wherein the pump comprises an adhesive patch and is a patch pump.

10. The insulin pump system of claim 9, wherein the pump further comprises a battery, and wherein the first device is a charger device configured to charge the battery.

11. An insulin pump system worn by a user and in wireless communication with a first device, the insulin pump system comprising:
a pump configured to pump insulin into the user based on first pump parameters;
memory configured to store computer-executable instructions; and
at least one computer processor configured to access memory and execute the computer-executable instructions to:
determine a computational operation is necessary to generate second pump parameters for operating the pump;
determine that a second device is to perform the computational operation;
send instructions to the first device to instruct the second device to perform the computational operation on the second device to generate the second pump parameters;
receive, after sending instructions to the first device, the second pump parameters from the first device, the second pump parameters generated by the second device and different than the first pump parameters; and
adjust, automatically, operation of the pump based on the second pump parameters upon receiving the second pump parameters.

12. The insulin pump system of claim 11, wherein the at least one computer processor is further configured to access memory and execute the computer executable instructions to send pump data corresponding to the computational operation to the first device and the second pump parameters are generated by the second device based at least in part on the pump data.

13. The insulin pump system of claim 11, wherein the first device is a mobile phone and the second device is a server and the first device and the at least one computer processor associated with the pump communicate via a first network type and the first device and the second device communicate via a second network type different than the first network type.

14. The insulin pump system of claim 11, wherein the at least one computer processor is further configured to access memory and execute the computer executable instructions to send authentication data to the first device.

15. The insulin pump system of claim 11, wherein the at least one computer processor is further configured to access memory and execute the computer executable instructions to determine to generate second pump parameters based on sensor data and/or user input data.

16. The insulin pump system of claim 11, wherein the at least one computer processor is further configured to access memory and execute the computer executable instructions to determine the second device corresponds to the computational operation.

17. The insulin pump system of claim 16, wherein the determination that the second device corresponds to the computational operation is based on a table comprising a plurality of computational operations and associated devices for executing a respective one of the plurality of computational operations.

18. The insulin pump system of claim 11, further comprising second computer executable instructions configured to be executed by at least one computer processor on the first device to cause the first device to send second instructions to the second device to cause the second device to execute the computational operation to generate the second pump parameters.

19. The insulin pump system of claim 18, further comprising third computer executable instructions configured to be executed by at least one computer processor on the second device to cause the second device to execute the computational operation to generate the second pump parameters.

20. The insulin pump system of claim 11, wherein the pump further comprises a battery, and wherein the first device is a charger device configured to charge the battery and wherein the pump comprises an adhesive patch and is a patch pump.
